(19) United States
(12) Patent Application Publication
(10) Pub. No.: US 2014/0228667 A1
(43) Pub. Date: Aug. 14, 2014

(54) DETERMINING LESIONS IN IMAGE DATA OF AN EXAMINATION OBJECT

(71) Applicants: Peter DANKERL, Baiersdorf (DE); Matthias HAMMON, Nuremberg (DE); Michael KELM, Erlangen (DE); Michael SÜHLING, Erlangen (DE); Alexey TSYMBAL, Erlangen (DE); Michael WELS, Bamberg (DE); Andreas WIMMER, Forchheim (DE)

(72) Inventors: Peter DANKERL, Baiersdorf (DE); Matthias HAMMON, Nuremberg (DE); Michael KELM, Erlangen (DE); Michael SÜHLING, Erlangen (DE); Alexey TSYMBAL, Erlangen (DE); Michael WELS, Bamberg (DE); Andreas WIMMER, Forchheim (DE)

(73) Assignee: SIEMENS AKTIENGESELLSCHAFT, Munich (DE)

(21) Appl. No.: 14/176,271

(22) Filed: Feb. 10, 2014

(30) Foreign Application Priority Data
Feb. 12, 2013 (EP) 13154927.1

(51) Int. Cl. A61B 5/00

(52) U.S. Cl. CPC A61B 5/4887
USPC 600/407

(57) ABSTRACT

A method in radiological imaging for determining lesions in image data of an examination object is described. In an embodiment, the method includes determining anatomical structures by hierarchical breakdown of the image data of the examination object. The method furthermore includes image data analysis for localizing lesion candidates in the anatomical structures. Moreover, the method also includes determining the lesions by evaluating and filtering the lesion candidates. Moreover, an image processing workstation in radiological imaging for determining lesions in image data of an examination object and an imaging apparatus are described.

### PRIORITY STATEMENT

The present application hereby claims priority under 35 U.S.C. §119 to European patent application number EP EP13154927.1 filed Feb. 12, 2013, the entire contents of which are hereby incorporated herein by reference.

### FIELD

At least one embodiment of the invention generally relates to a method in radiological imaging for determining lesions in image data of an examination object. Moreover, at least one embodiment of the invention generally relates to an image-processing workstation in radiological imaging for determining lesions in image data of an examination object and/or to an imaging apparatus and/or a computer program product.

### BACKGROUND

These days imaging systems from medical engineering play an important role in the examination of patients. The representations, produced by the imaging systems, of the inner organs and structures of the patient are used for screening, for biopsies, in the diagnosis of the causes of disease, for planning surgery, when carrying out surgery or else for preparing therapeutic measures. Examples of such imaging systems include ultrasound systems, x-ray devices, x-ray computed tomography (CT) systems, positron emission tomography (PET) systems, single-photon emission computed tomography (SPECT) systems and magnetic resonance imaging (MRI) systems.

A field of application for imaging systems which continues to gain importance lies in examinations for cancer screening and supporting therapeutic measures when treating cancerous diseases. However, despite improved imaging equipment and an increased functionality of the associated software means, the treatment of patients with a cancerous disease remains a great challenge. Studies in the European Union confirm that cancerous diseases as cause of death of patients are not decreasing, but are even on the increase in recent years for certain types of cancer, such as pancreatic cancer or lung cancer in women.

In general, early detection by specialist medical staff is decisive in all cancers for successful treatment of the cancerous disease. Here, [E. A. Krupinski, “Computer-aided detection in clinical environment: benefits and challenges for radiologists”, Radiology, 231, 2004, 7-9] could already show that even the most experienced radiologists achieve better results in the detection of cancerous diseases when interpreting radiological image data as a result of suitable computer or software support. Apart from the identification of general injury to anatomical structures of a patient, the so-called lesions, it was found that the assessment and risk evaluation of lesions is difficult and susceptible to error, particularly if these are lesions with relatively small dimensions, even though precisely the early detection of relatively small lesions, that is to say e.g. cancerous disease which is at an early stage, can decisively contribute to curing a patient.

Accordingly, the detection and evaluation of lesions has great importance in medical practice. It is known from estimates that the detection and evaluation of lesions makes up more than 60% of the diagnostic activity of the specialist medical staff. However, not every lesion can be evaluated reliably directly by the imaging system that was used for detecting a lesion.

Therefore, there is a need for solutions by which lesions can be detected and evaluated in a reliable manner, which is also expedient in terms of time used and costs. In particular, there is a need for solutions for early and reliable identification of relatively small, malignant lesions in the case of cancerous disease since there is a drop in possibilities for cure (and the treatment complexity increases) the later a malignant lesion is identified in the body of the patient.

Currently, individual technical solutions are known, by which lesions in the body of a patient can be detected, for example the Syngo® suite by Siemens AG or the prototype for detecting metastases in CT image data of the vertebral column, described in [M. Wels, et al., “Multi-stage osteolytic spinal bone lesion detection from CT data with internal sensitivity control”, Proc. SPIE Medical Imaging, 2012].

Here, such systems and solutions in the prior art are isolated, i.e. optimized for a specific anatomical structure or body region and specialized for the detection of lesions of a specific type of cancerous disease. As a result of this specific alignment of the known solutions, there is often no success in using these for detecting metastases in the body of a patient early if these metastases originate from a primary cancerous disease in a different anatomical structure or different body region. This applies in particular to the frequent metastases or malignant lesions in the skeleton of a patient, which are caused by a primary cancerous disease in the lung, in the breast or in the colon.

### SUMMARY

At least one embodiment of the present invention specifies a method and/or apparatus which lessens or even avoids at least one of the above-described disadvantages of the prior art in the detection of lesions and which is not restricted to a specific body region or a specific anatomical structure of a patient.

A method and an image-processing workstation are disclosed.

The method according to at least one embodiment of the invention in radiological imaging for determining lesions in image data of an examination object comprises a first step, in which anatomical structures are determined by hierarchical breakdown of the image data of the examination object. In a second step of the method according to at least one embodiment of the invention, there is an image data analysis for localizing lesion candidates in the previously determined anatomical structures. In a third step of the method according to at least one embodiment of the invention, lesions are determined by evaluating and filtering the lesion candidates.

An imaging apparatus according to at least one embodiment of the invention, for example an ultrasound system, an x-ray device, a mammography system, an x-ray computed tomography (CT) system, a positron emission tomography (PET) system, a single-photon emission computed tomography (SPECT) system or a magnetic resonance imaging (MRI) system is characterized by an image-processing workstation according to at least one embodiment of the invention.

A technical implementation of the methods according to embodiments of the invention can be brought about in very different ways. In particular, it is feasible that an implementation is carried out at least in part with the aid of electrical circuits such as ASICs (application-specific integrated circuits), FPGAs (field programmable gate arrays) or PLAs (programmable logic arrays).

### BRIEF DESCRIPTION OF THE DRAWINGS

The invention will be once again explained in more detail in the following text on the basis of exemplary embodiments, with reference being made to the attached figures. In detail:

FIG. 1 shows a schematic depiction of an embodiment of the method according to the invention,

FIG. 2 shows a plurality of image data examples for the normalization, according to an embodiment of the invention, of anatomical structures,

FIG. 3 shows a plurality of image data examples for false positive classifications of lesions,

FIG. 4 shows three image data examples for the rule-based classification,

FIG. 5 shows measurement data of the spatial distribution of malignant blastic lesions and benign abnormalities in normalized vertebrae of the vertebral column,

FIG. 6 shows two examples of the sensitivity (true positive rate) as a function of the number of false positives per unit volume during the lesion detection,

FIG. 7 shows an image data example of the human hand and

FIG. 8 shows an embodiment of the image-processing workstation according to the invention.

### DETAILED DESCRIPTION OF THE EXAMPLE EMBODIMENTS

The present invention will be further described in detail in conjunction with the accompanying drawings and embodiments. It should be understood that the particular embodiments described herein are only used to illustrate the present invention but not to limit the present invention.

Accordingly, while example embodiments of the invention are capable of various modifications and alternative forms, embodiments thereof are shown by way of example in the drawings and will herein be described in detail. It should be understood, however, that there is no intent to limit example embodiments of the present invention to the particular forms disclosed. On the contrary, example embodiments are to cover all modifications, equivalents, and alternatives falling within the scope of the invention. Like numbers refer to like elements throughout the description of the figures.

Specific structural and functional details disclosed herein are merely representative for purposes of describing example embodiments of the present invention. This invention may, however, be embodied in many alternate forms and should not be construed as limited to only the embodiments set forth herein.

It will be understood that, although the terms first, second, etc. may be used herein to describe various elements, these elements should not be limited by these terms. These terms are only used to distinguish one element from another. For example, a first element could be termed a second element, and, similarly, a second element could be termed a first element, without departing from the scope of example embodiments of the present invention. As used herein, the term “and/or,” includes any and all combinations of one or more of the associated listed items.

It will be understood that when an element is referred to as being “connected,” or “coupled,” to another element, it can be directly connected or coupled to the other element or intervening elements may be present. In contrast, when an element is referred to as being “directly connected,” or “directly coupled,” to another element, there are no intervening elements present. Other words used to describe the relationship between elements should be interpreted in a like fashion (e.g., “between,” versus “directly between,” “adjacent,” versus “directly adjacent,” etc.).

The terminology used herein is for the purpose of describing particular embodiments only and is not intended to be limiting of example embodiments of the invention. As used herein, the singular forms “a,” “an,” and “the,” are intended to include the plural forms as well, unless the context clearly indicates otherwise. As used herein, the terms “and/or” and “at least one of” include any and all combinations of one or more of the associated listed items. It will be further understood that the terms “comprises,” “comprising,” “includes,” and/or “including,” when used herein, specify the presence of stated features, integers, steps, operations, elements, and/or components, but do not preclude the presence or addition of one or more other features, integers, steps, operations, elements, components, and/or groups thereof.

It should also be noted that in some alternative implementations, the functions/acts noted may occur out of the order noted in the figures. For example, two figures shown in succession may in fact be executed substantially concurrently or may sometimes be executed in the reverse order, depending upon the functionality/acts involved.

Unless otherwise defined, all terms (including technical and scientific terms) used herein have the same meaning as commonly understood by one of ordinary skill in the art to which example embodiments belong. It will be further understood that terms, e.g., those defined in commonly used dictionaries, should be interpreted as having a meaning that is consistent with their meaning in the context of the relevant art and will not be interpreted in an idealized or overly formal sense unless expressly so defined herein.

Spatially relative terms, such as “beneath”, “below”, “lower”, “above”, “upper”, and the like, may be used herein for ease of description to describe one element or feature's relationship to another element(s) or feature(s) as illustrated in the figures. It will be understood that the spatially relative terms are intended to encompass different orientations of the device in use or operation in addition to the orientation depicted in the figures. For example, if the device in the figures is turned over, elements described as “below” or “beneath” other elements or features would then be oriented “above” the other elements or features. Thus, term such as “below” can encompass both an orientation of above and below. The device may be otherwise oriented (rotated 90 degrees or at other orientations) and the spatially relative descriptors used herein are interpreted accordingly.

Although the terms first, second, etc. may be used herein to describe various elements, components, regions, layers and/or sections, it should be understood that these elements, components, regions, layers and/or sections should not be limited by these terms. These terms are used only to distinguish one element, component, region, layer, or section from another region, layer, or section. Thus, a first element, component, region, layer, or section discussed below could be termed a second element, component, region, layer, or section without departing from the teachings of the present invention.

The method according to at least one embodiment of the invention in radiological imaging for determining lesions in image data of an examination object comprises a first step, in which anatomical structures are determined by hierarchical breakdown of the image data of the examination object. Here, and in the following, the term “examination object” or “patient” represents a human undergoing medical treatment or an animal undergoing medical treatment. Here, this also includes examination objects that are not diseased, i.e. also humans in which image data are produced for prevention, e.g. during preventative screening for cancer prevention. In the following, the terms “examination object” and “patient” are used synonymously and without restricting the invention. Moreover, the invention makes no distinction between female and male patients and “patient” (which is masculine in German) is used uniformly throughout.

The image data could have been produced by a measurement or image data recording using a system from radiological imaging. By way of example, the image data can be a two-dimensional image of a body region of the examination object, wherein the image was recorded using an x-ray apparatus which is conventional in medical practice. Likewise, three-dimensional recording methods for producing the image data are also feasible, i.e., for example, methods using CT, PET, MRI systems or digital tomosynthesis methods, as are used e.g. in mammographic diagnostics.

Here, in addition to the bones of the skeleton, the anatomical structures of the examination object are also non-bone-like structures, such as e.g. organs, tissue, muscles, connective tissue, layers of skin, nerves or blood vessels. As a result of a hierarchical breakdown or else segmentation of the radiological image data, individual anatomical structures are, firstly, identified in the image data. That is to say that e.g. the liver or the cervical vertebrae of a patient are localized in the image data.

Moreover, the spatial relationship of the individual anatomical structures in relation to one another is determined by the hierarchical breakdown of the image data. Such a breakdown of the radiological image data enables reliable and efficient navigation to the anatomical structures in the further steps of the method according to the invention. The result of the hierarchical breakdown can likewise also support the specialist medical staff during a navigation, which follows the method, to the lesions determined by the method.

Here, the hierarchical breakdown can, at least in portions of the examination object, be carried out in parallel. By way of example, the breakdown of the image data in the region of the right arm can take place in parallel with a breakdown of the image data in the region of the left arm. This reduces the time required for carrying out the method step. Furthermore, a largely automated embodiment of the method step is possible by appropriate computer-assisted means and methods.

Within the method according to at least one embodiment of the invention, the hierarchical breakdown can also be supported and complemented by a knowledge base for anatomical structures. In particular, a so-called ontology or ontological knowledge base can provide information in respect of the properties of individual anatomical structures and in respect of the relationships between the individual anatomical structures. Here, the ontology is preferably available in a machine-readable representation, which is suitable for a computer-assisted embodiment of the hierarchical breakdown.

In a second step of the method according to at least one embodiment of the invention, there is an image data analysis for localizing lesion candidates in the previously determined anatomical structures. Such an image data analysis examines the image data for deviations from reference data, i.e. anatomical abnormalities, which can be an indication of the presence of a lesion. Therefore, this results in lesion candidates. By way of example, a specific grayscale value step in radiological image data can indicate the presence of a lesion. Once again, a largely automated embodiment of this method step is possible by appropriate computer-assisted devices and methods, and also preferable.

In a third step of the method according to at least one embodiment of the invention, lesions are determined by evaluating and filtering the lesion candidates. Here, the lesion candidates can be evaluated by suitable criteria and subsequently filtered due to this evaluation. The lesions remaining after this filtering are subsequently available for further analysis and evaluation by the specialist medical staff. Here, as a result for the determined lesions, the filtering, in addition to a subset of the lesion candidates, also can have all lesion candidates or else none of the lesion candidates.

By way of example, filtering may take place dependent on the size of the lesions. As a result of this, the lesions can be categorized in size classes. In particular, an assessment of the course of the disease is made possible in the case of a multiple application of the method to a patient by evaluating the spread of lesions and the size growth thereof. The filtering of the lesion candidates preferably takes place with the aid of specialist medical knowledge, wherein the specialist knowledge is preferably represented in a representation that is suitable for computer-assisted processing.

It is understood that the lesions determined by at least one embodiment of the method do not necessarily correspond exactly to the actual lesions situated in the body of the patient. As a result of imprecision in the production of the radiological image data and the predictive nature of the method, the lesions determined by the method are only specifications in relation to a body region, in which a lesion is expected with a certain probability.

However, the properties of the method according to at least one embodiment of the invention and the embodiments thereof are intended to contribute to the probability of a correct detection of a lesion being improved compared to the prior art. A precise statement about or confirmation of the presence of a lesion and the properties thereof then can be made e.g. by a surgical intervention in the patient and optionally a subsequent histology. However, using the method according to at least one embodiment of the invention, it is to be expected that unnecessary and undesired surgical interventions for cancer prevention, so-called biopsies, are avoided or reduced. A largely automated embodiment of the method step is also possible for the third method step by appropriate computer-assisted devices and methods.

The method according to at least one embodiment of the invention is distinguished by the fact that it can be applied to all body regions of a patient and is not restricted to one body region. As a result, it is also possible to detect malignant lesions at locations at which a radiologist may not have expected them. This in turn can contribute to an early detection and, connected therewith, improved prospects of cure and a reduced number of burdensome follow-up examinations.

Furthermore, the method according to at least one embodiment of the invention is not set to a specific type of lesions, type of cancer or form of metastasis. Hence, a method can be made available to specialist medical staff, which method is operable in a uniform manner, independent of the cancerous disease or the body region, and has a broad spectrum of application. Ultimately, this enables an efficient operation, which is not susceptible to errors, by the specialist medical staff. Moreover, the method resorts to specialist medical knowledge or knowledge bases, and so the desired high reliability when determining lesions can set in. Moreover, there can be unburdening of the specialist medical staff by the partial or else complete automation of the method by way of suitable computer assistance.

The method according to at least one embodiment of the invention is furthermore distinguished by the fact that the three method steps can evaluate the image data of the examination object at different levels of abstraction, and hence an integrated and comprehensive determination of lesions is achieved. By way of example, the image data analysis for localizing lesion candidates is more likely to resort to simple image processing methods at a lower level of abstraction, like the aforementioned evaluation of grayscale values in the image data. By contrast, determining the lesions by evaluating and filtering is more likely to use methods and knowledge which derives from overarching specialist medical knowledge, such as the knowledge about certain clinical pictures. Therefore, this method step is more likely to be associated with a higher level of abstraction.

An image-processing workstation according to at least one embodiment of the invention in radiological imaging for determining lesions in image data of an examination object comprises a structure determination apparatus for determining anatomical structures by hierarchical breakdown of the image data of the examination object. Furthermore, the image-processing workstation according to at least one embodiment of the invention comprises an image data analysis apparatus for localizing lesion candidates in the anatomical structures. Moreover, the image-processing workstation according to at least one embodiment of the invention comprises a lesion determination apparatus for determining the lesions by evaluating and filtering the lesion candidates.

Here, the structure determination apparatus according to at least one embodiment of the invention, the image data analysis apparatus according to at least one embodiment of the invention or the lesion determination apparatus according to at least one embodiment of the invention can be partly or wholly embodied by hardware components, for example using semiconductor components such as ASICs (application-specific integrated circuits), FPGAs (field programmable gate arrays) or PLAs (programmable logic arrays). Moreover, a computer program product, which can be loaded directly into a memory of a programmable imaging apparatus, can execute the methods according to embodiments of the invention, at least in part, using program code means, when the computer program product is executed in the imaging apparatus.

The dependent claims each contain particularly advantageous embodiments and developments of the invention, wherein an image-processing workstation, according to at least one embodiment of the invention, for determining lesions in image data of an examination object can have analogous developments to the dependent claims of the method, according to at least one embodiment of the invention, for determining lesions in image data of an examination object.

Since the method according to at least one embodiment of the invention comprises a method step for determining anatomical structures, the further method steps can be controlled in an advantageous fashion by the available data in relation to the anatomical structures. Preferably, the method is embodied in such a way that localizing the lesion candidates and/or determining the lesions is/are controlled by specific features of the anatomical structures. By way of example, a detected osteolytic lesion candidate, which is situated in the vicinity of a basivertebral vein within a vertebra of the patient, can be classified as physiological, i.e. non-pathological, and eliminated from the set of lesion candidates. Using such a control of the method according to the invention as a result of knowledge of the anatomical structures, there is an improved efficiency and a reduced number of incorrectly predicted malignant lesions.

As a further example, sclerotic degenerations and therefore benign abnormalities can be mentioned, which are known for occurring in the vicinity of the cortical bone tissue. Accordingly, these abnormalities advantageously can be eliminated from the set of lesion candidates if the associated structure is known. That is to say that the preceding hierarchical breakdown (segmentation) results in a contribution to identifying and eliminating incorrect lesion determinations.

The method according to at least one embodiment of the invention can be likewise embodied in such a way that localizing the lesion candidates and/or determining the lesions is/are controlled by specific features of a specific body region of the examination object. The advantages emerging from this embodiment are comparable to the aforementioned advantages in the case of a control by specific features of the anatomical structures.

In a preferred embodiment, the method according to at least one embodiment of the invention is characterized in that a number of rule-based classifiers are used for localizing the lesion candidates and/or for determining the lesions, preferably a number of knowledge-based classifiers, which were trained using image data of other examination objects. In so doing, a “number” refers here and in the following to a positive natural number greater than zero. In particular, the classifiers can be embodied in such a way that they are controlled by the specific features of the anatomical structures. Here, a classifier or else a classifying method assigns one or more classes to an object in the radiological image data as a result of the properties thereof.

A number of classifiers can be applied to distinguish between benign changes in the bone structure of a patient and malignant lesions. Examples of this include the distinction between osteophytes and degenerative sclerosis of malignant, blastic lesions or the distinction between benign abnormalities such as osteopenia, osteoporosis, hemangioma or Schmorl's node and malignant lytic lesions. Here, a knowledge-based classifier can be distinguished by rules which were produced or optimized using the available training image data from other patients. In particular, these can be training image data which were annotated by an expert in the field of medicine, for example by marking the image data and a corresponding classification.

Furthermore, additional properties of the respective anatomical structure, which can be used in an advantageous manner for the specialist medical staff and the further treatment, can be determined by the method according to at least one embodiment of the invention. By way of example, if an osteolytic bone lesion is determined in a vertebra by the method according to at least one embodiment of the invention, then the method can generate a note in respect of a possible risk of a vertebral fracture for the relevant vertebra. In so doing, use can be made of rule-based classifiers and/or knowledge-based classifiers, which were trained by data from other patients, wherein these data contain information in this case in respect of the occurrence of vertebral fractures. Such generation of additional information is also referred to as a subordinate classification or a secondary classification system.

In a preferred embodiment, the method according to at least one embodiment of the invention is characterized in that the number of rule-based classifiers comprises a number of false positive classifiers, preferably knowledge-based false positive classifiers, which were trained by false positive image data from other examination objects. Here, a “false positive classifier” is understood to mean a classifier which has as its goal to reevaluate the (positive) detections (candidates) of a preceding classifier in order thereby to exclude false positives, without in the process reducing the number of correctly detected lesions (i.e. the “true positives”). By way of example, if malignant lesions are detected in the vertebral column, there can be an application of a number of false positive classifiers which are based on rules like the following rule: “A lytic lesion is classified as a benign vertebral lesion if this is a lysis with low contrast AND if it is situated centrally in the rear plane of a vertebra”. Using this rule, lesions which would otherwise lead to a false positive result, i.e. the detection of an in fact benign lesion as a malignant lesion, would advantageously be removed from the set of lesion candidates.

In a further embodiment, the method according to at least one embodiment of the invention is characterized in that, for determining the anatomical structures, a number of anatomical atlases are used and/or a number of anatomical landmarks are determined in the image data. Here, the anatomical atlases support the determination of the anatomical structures and establish a spatial or content-based relationship between anatomical structures. Here, anatomical landmarks generally are anatomical conditions of the examination object, which have particular properties or are easy to identify in radiological image data. Examples of anatomical landmarks include the corners of the eye, the tip of the nose, the nipple (papilla), a specific vertebra of the vertebral column, or the anterior commisure (AC) and posterior commisure (PC) of the brain.

By way of example, in the method according to at least one embodiment of the invention, all bones in the skeleton can be determined in the image data during the hierarchical breakdown with the aid of anatomical atlases and/or anatomical landmarks and a classification of all bones can be subsequently identified by way of a suitable anatomical ontology. Such a classification can then form the basis for the further steps of the method, i.e. control determining of the lesion candidates and/or the lesions. Moreover, a classification of the bones in the skeleton of the patient can also serve to identify and classify adjacent anatomical structures, such as internal or subcutaneous fat, muscles, organs or vessels, in the image data.

A further embodiment of the method according to the invention is characterized in that the method comprises a method step for determining pathological abnormalities in the anatomical structures.

In a particularly preferred embodiment of the method according to the invention, analyzing image data for localizing lesion candidates is predominantly carried out on the basis of pixels and/or voxels. Here, the term “pixel” describes an image point in two-dimensional radiological image data, whereas a “voxel” (“volumetric pixel”) describes an image point in three-dimensional radiological image data. Particularly in the case of CT imaging, the pixels or voxels in the image data specify how strong the x-ray radiation is attenuated when passing through the body of the patient. Here, as a scale, use is often made of the so-called Hounsfield scale, wherein, in the case of a graphical output, the Hounsfield scale values are represented by a grayscale value scale. Bone structures are then usually displayed more brightly than other anatomical structures since bone structures attenuate the x-ray radiation more strongly.

Accordingly, in this embodiment of the method according to the invention, the image data analysis in the case of CT image data would be predominantly based on an analysis of the Hounsfield values. By way of example, a lesion candidate is identified if an unexpectedly high contrast difference occurs in the image data. Here, the analysis in turn can be controlled by information about the respectively analyzed anatomical structure since the significance of contrast differences for determining lesions depends on the respective anatomical structure. It is understood that an image data analysis for determining lesion candidates, which is predominantly carried out on the basis of pixels or voxels, is particularly suitable for a computer-assisted, automatic embodiment.

The method according to at least one embodiment of the invention can furthermore preferably comprise a method step for normalizing, more particularly for spatially normalizing, the anatomical structures. A normalization is advantageous if it relates to anatomical structures which occur a number of times in the human body and which often only differ slightly from one another. Such anatomical structures include the vertebral bones in the vertebral column, the phalanges in the hand, the phalanges in the foot, but also, for example, the mutually corresponding upper arm and femoral bones. Here, the latter are very similar in respect of the bone density, the structure, the perfusion and the physiological functionality thereof. Furthermore, a normalization also lends itself to arterial vessels close to the heart and to muscle groups that are similar to one another.

By way of example, a normalization of the image data can comprise e.g. a rotation or scaling of the image data of the anatomical structure. As a result of such a normalization of the anatomical structures, the technical implementation of the method according to at least one embodiment of the invention becomes easier since anatomy-specific method steps need not be carried out for each individual anatomical structure. Rather, it is possible, for example, to specify classifiers which can be applied to a majority of the vertebrae in the vertebral column after an appropriate normalization. If these here are knowledge-based classifiers, this embodiment of the method according to the invention can also increase the quality of the classifiers since the training image data then comprise not only the respective anatomical structure but all anatomical structures that are similar to one another. A classifier for determining lesions in a vertebra of the vertebral column can accordingly be trained using a very large number of vertebrae, as a result of which the reliability of the lesion determination improves.

Here, the aforementioned embodiment of the method according to the invention is based, inter alia, on the discovery that the lesions often occur at the same spatial regions of the anatomical structure in the case of anatomically similar structures. This clinically confirmed discovery applies to both malignant and benign lesions, and so such an embodiment can also be used advantageously for false positive detectors. Moreover, a normalization in general can also be carried out for all bone structures, for example a normalization of the Hounsfield values in CT imaging. As a result, undesired imaging- or patient-specific deviations can be reduced or avoided when determining the lesions.

A particularly preferred embodiment of the method according to the invention is characterized in that the method is controlled by specific features of the examination object. Such features can comprise both the patient-specific physiology and physiognomy, and also clinical pictures which have direct or else only indirect reference for determining the lesions. By way of example, the threshold for detecting lesion candidates in the bone structures of a patient can be increased if it is known that the patient suffers from osteoporosis. This is advantageous because osteoporosis often leads to bone abnormalities which have a similarity in radiological image data to malignant lesions. As a result, the number of false positive lesion determinations can be reduced by such an embodiment.

There is direct reference to determining lesions if the type of a primary cancerous disease is already known. In this case, the method can advantageously be restricted to anatomical structures in which malignant lesions or metastases are to be expected for the respective type of cancer. By way of example, it is known that the metastases in the case of illness due to a primary prostate cancer only occur in the bone structures of the patient. Accordingly, the method according to the invention for determining lesions can be restricted to the bone structures in this case. In general, the method according to the invention in this embodiment can also directly determine patient-specific features from the radiological image data, for example the average bone density or the stature of the patient or else the type of primary cancerous disease.

In addition to determining lesions, the method according to at least one embodiment of the invention preferably can comprise a separating classification of the lesions into benign lesions and malignant lesions. It is once again understood that such a classification by the method may be susceptible to errors since only a surgical intervention and a subsequent histological examination can obtain complete certainty about the type of lesion. Therefore, the aforementioned classification is a statement relating to the probability that a lesion is benign or malignant. Despite this imprecision that cannot be excluded, such an embodiment of the invention, however, is advantageous since this renders many unnecessary surgical interventions avoidable.

In addition to the separating classification of the lesions, the method according to at least one embodiment of the invention may also be characterized in that it determines the change in lesion dimensions and the numerical and spatial spread of the lesions by comparisons with previous radiological image data from the same patient. In particular, it is possible to assess in this case the extent to which a patient responds to a therapeutic measure. The method can likewise also produce a therapy suggestion, optionally also by using radiological image data from other patients.

Furthermore, the method according to at least one embodiment of the invention can be characterized in that determining the lesions comprises a separating classification of the lesions into blastic and lytic lesions. Here, such a classification can be based on the aforementioned grayscale value steps during CT imaging and can also be carried out automatically since lytic and blastic lesions often differ not insubstantially in terms of the grayscale values.

The image data of the examination object particularly preferably comprise a whole-body image data record during the application of the method according to at least one embodiment of the invention. The use of a whole-body image data record advantageously contributes to lesions also being able to be determined in those body regions of the patient in which a specialist medical expert would not have expected any lesions. Accordingly, the probability of the lesion detection improves. At least for the hierarchical breakdown of CT whole-body image data, first methods are already known from [S. Seifert, et al., “Hierarchical parsing and semantic navigation of full body CT data”, Proc. SPIE Medical Imaging, 2008], and these are suitable as an initial point for determining lesions according to at least one embodiment of the invention. As a result of the fact that the method according to the invention is not restricted to a specific body region or a specific type of cancer, it is possible, particularly when using whole-body image data, to derive a universal, multifunctional computer-assisted lesion determination system and lesion characterization system from the features of at least one embodiment of the invention.

In a further embodiment, the method according to at least one embodiment of the invention is characterized in that the method determines lesions in the skeleton of the examination object, preferably in the vertebral column of the examination object. Lesions in the skeleton of the patient occur in conjunction with a multiplicity of cancerous diseases, for example in the case of cancerous diseases of the prostate, of the breast, of the thyroid, of the kidneys, of the pancreas and of the lung. They are often accompanied by bone diseases such as hypercalcemia, bone fractures, spinal compression, bone pain and similar pains, which are caused by a compression of the nerves. Too late detection of lesions in the bone structures can therefore lead to crippling pain, immobility, neurological disabilities and paralysis. Metastases in the skeleton of the patient often lead to only a short survival time with a median value of only 6 months.

Furthermore, it is known that lesions in bones may occur in lytic form, in blastic form or else in a mixed form, as a result of which determining the lesions becomes more difficult. This applies in particular because lesions in the bones can often only be distinguished with difficulties in the image data from the symptoms of other diseases, such as osteophytes or osteoporosis. A method according to at least one embodiment of the invention adapted to determining lesions in bone structures can therefore deliver a substantial contribution to an improved treatment of the patients and to a reduction in terms of time and costs. This applies in particular to a combination of this embodiment with the aforementioned use of whole-body image data.

The method is preferably characterized in that the image data of the examination object are determined using a computed tomography scanner, a magnetic resonance imaging scanner or a positron emission tomography scanner.

An imaging apparatus according to at least one embodiment of the invention, for example an ultrasound system, an x-ray device, a mammography system, an x-ray computed tomography (CT) system, a positron emission tomography (PET) system, a single-photon emission computed tomography (SPECT) system or a magnetic resonance imaging (MRI) system is characterized by an image-processing workstation according to at least one embodiment of the invention.

A technical implementation of the methods according to embodiments of the invention can be brought about in very different ways. In particular, it is feasible that an implementation is carried out at least in part with the aid of electrical circuits such as ASICs (application-specific integrated circuits), FPGAs (field programmable gate arrays) or PLAs (programmable logic arrays).

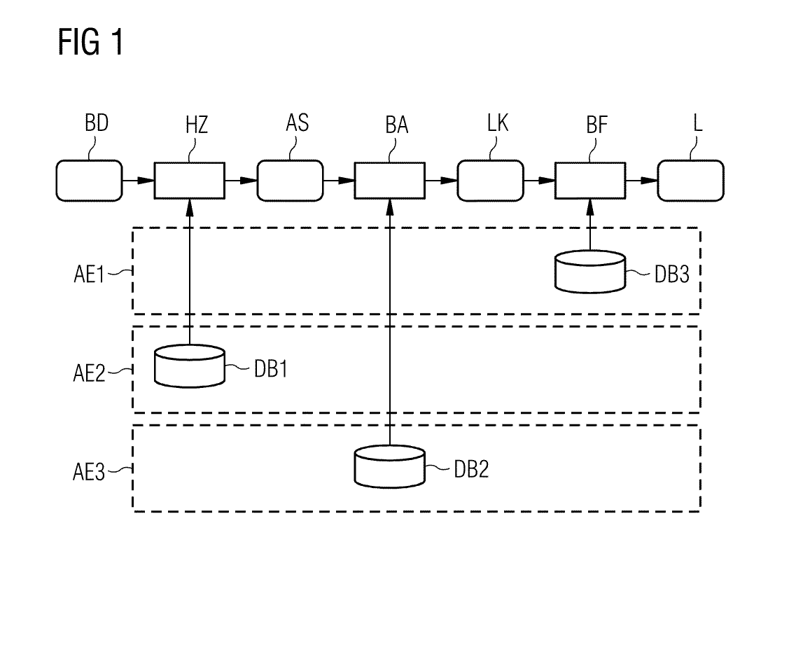

FIG. 1 shows a schematic representation of an embodiment of the method according to the invention, which determines lesions L in image data BD of an examination object. Proceeding from the image data BD, there is a hierarchical breakdown HZ or segmentation into anatomical structures AS using the database DB1. Here, information for identifying anatomical structures can be stored in the database DB1, i.e., for example, characterizing properties of anatomical landmarks or else information from anatomical atlases. In this case, the anatomical structures AS can also comprise a classification (ontology, taxonomy) of the anatomical structures and the spatial and functional dependencies thereof with respect to one another.

A subsequent image data analysis BA determines lesion candidates LK using a further database DB2. Here, this database DB2 can, inter alia, provide detectors which determine lesion candidates LK in the anatomical structures AS. Here, both general detectors and detectors that are optimized to specific anatomical structures AS are feasible, for example detectors for the vertebral bones of the vertebral column. The image data analysis BA can be preceded by pre-processing, which for example carries out a normalization of the anatomical structures AS. Furthermore, the image data analysis can also be followed by post-processing, in which there is first filtering of the lesion candidates LK or in which the lesion candidates LK are assigned a probability. The post-processing may in this case be controlled by e.g. other features, such as a mean, patient-specific bone density.

By way of the subsequent evaluating and filtering BF, there is a determination of those lesion candidates LK of the totality thereof which are output as lesions L as result of the lesion determination by the method according to the invention. Here, evaluating and filtering BF employs data and knowledge stored in the database DB3. In particular, the database DB3 can comprise rule-based or knowledge-based classifiers which verify, evaluate and, on the basis of the evaluation, filter the established lesion candidates LK and output these as determined lesions L. Here, in particular, there can also be further classification of the lesions L, for example a separation into benign and malignant lesions or a separation into blastic and lytic lesions. Moreover, use can be made of subordinate classifiers or secondary classifiers, which determine further properties or features of the lesions L, for example probability for the occurrence of bone fractures in the relevant anatomical structures AS.

In the embodiment of the method according to the invention depicted here, the databases DB1, DB2 and DB3 are classified in different abstraction levels or tiers AE1, AE2 and AE3. Here, the abstraction level AE1 describes the highest abstraction and the abstraction tier AE3 describes the lowest abstraction. By way of example, the image data analysis BA for determining the lesion candidates LK is often more likely to be a method step based on elementary image processing methods, inter alia on the analysis of grayscale values and the spatial contrasts thereof. Accordingly, the data stored in the database DB2 and the stored knowledge is information with a relatively low abstraction level in the abstraction tier AE3.

In contrast thereto, the data and the knowledge stored in the database DB3 are arranged on the highest abstraction tier AE1 in the embodiment shown here. This emerges from the fact that, when determining lesions L, use is made of rule- and knowledge-based classifiers which are based on medical knowledge and often many years' worth of clinical experience. These also include the aforementioned false positive classifiers.

The data stored in the database DB1 and the knowledge stored in the database DB1 are classified in the middle abstraction tier AE2 since, firstly, use is made of elementary image processing methods such as the analysis of grayscale contrasts in the image data BD. Secondly, when determining the anatomical structures AS, use is also made of relatively abstract knowledge in respect of the spatial and functional dependencies of anatomical conditions.

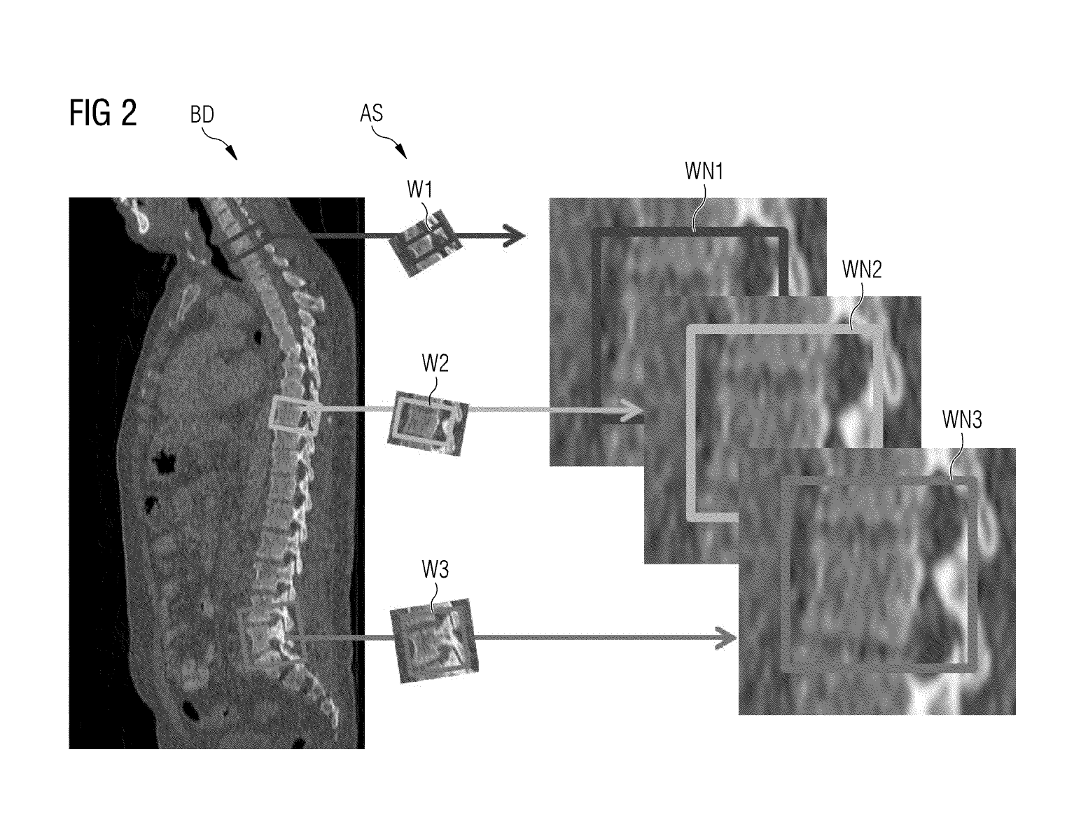

FIG. 2 reproduces a plurality of image data examples of the normalization, according to an embodiment of the invention, of anatomical structures AS. The image data BD of the examination object show a sagittal slice image through the body, substantially in the region of the vertebral column. In accordance with the method according to the invention or the image-processing workstation BS according to the invention, the vertebral column and, with it, the individual vertebrae of the vertebral column are identified as anatomical structures AS in the image data BD by hierarchical breakdown HZ. Subsequently, there is a normalization according to an embodiment of the invention of the anatomical structures AS, which is depicted in an exemplary manner in FIG. 2 on three vertebrae W1, W2, W3 of the vertebral column. The uppermost vertebra W1 is a cervical vertebra W1, the central vertebra W2 is a thoracic vertebra W2 and the lower vertebra W3 is a lumbar vertebra W3.

As can be identified from FIG. 2, the three vertebrae W1, W2, W3 differ in terms of their dimensions and their alignment. By way of a normalization step according to an embodiment of the invention, the image data BD of the three vertebrae W1, W2, W3 are imaged by rotation and scaling as vertebrae in a normalized representation WN1, WN2, WN3. This normalization renders it possible to specify uniform method steps for determining a lesion for a majority of the vertebrae in the vertebral column. It is not necessary to provide individual method steps, such as rule-based or knowledge-based classifiers, for each vertebra-specific dimension and alignment. Moreover, the normalization according to an embodiment of the invention offers the advantage that the number of training image data multiplies, as a result of which the quality of the method, more particularly the quality of knowledge-based classifiers, is improved. In addition to rotation and scaling, use can be made of further normalization steps, for example a normalization of the image data resolution or a normalization of the grayscale value ranges in the CT imaging.

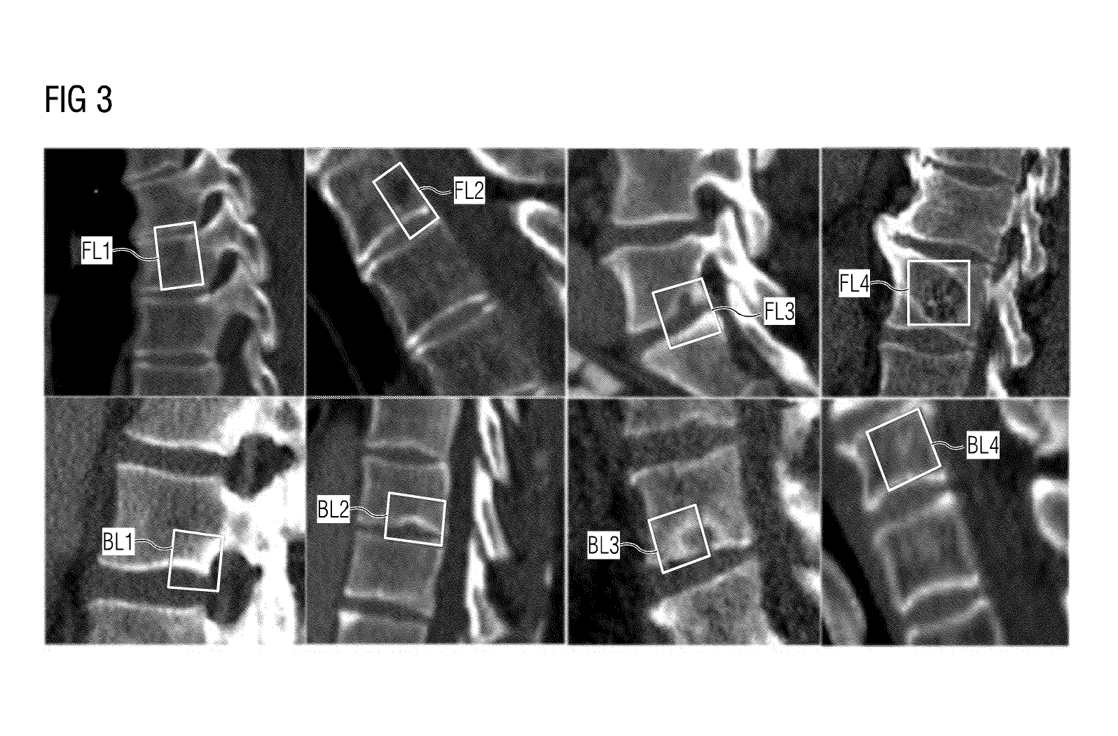

FIG. 3 reproduces a plurality of image data examples of false positive classifications of lesions L in the image data BD of the vertebral column of a patient. The upper four image data examples FL1, FL2, FL3, FL4 show lesions L which may possibly be identified as malignant lesions ML in lytic form by unsuitable lesion determination methods. The lower four image data examples BL1, BL2, BL3, BL4 show lesions which may possibly be identified as malignant lesions ML in blastic form by unsuitable lesion determination methods. In actual fact, the eight examples of false positive classifications FL1, FL2, FL3, FL4, BL1, BL2, BL3, BL4 are benign abnormalities.

In FIG. 3, the false positive classification examples FL1, FL2, FL3, FL4, BL1, BL2, BL3, BL4 are, from left to right, arranged respectively for the top and bottom row in decreasing frequency of the typical occurrence thereof. The lytic false positive classification FL1 is a region with osteoporosis, FL2 is a basivertebral vein, FL3 is a Schmorl's node and FL4 is a hemangioma. The blastic false positive classification BL1 is an osteophyte, BL2 is degenerative sclerosis and BL3 is a Schmorl's node. By contrast, the blastic false positive classification BL4 represents an erroneous classification by an unsuitable lesion determination method, caused by image data artifacts.

Such unwanted false positive classifications can be reduced or avoided by applying the method according to an embodiment of the invention or the image-processing workstation BS according to the invention, particularly if use is made of rule-based and/or knowledge-based classifiers, which model specialist medical knowledge or clinical experience or which were trained by training image data records, like the false positive classifications FL1, FL2, FL3, FL4, BL1, BL2, BL3, BL4 shown in FIG. 3.

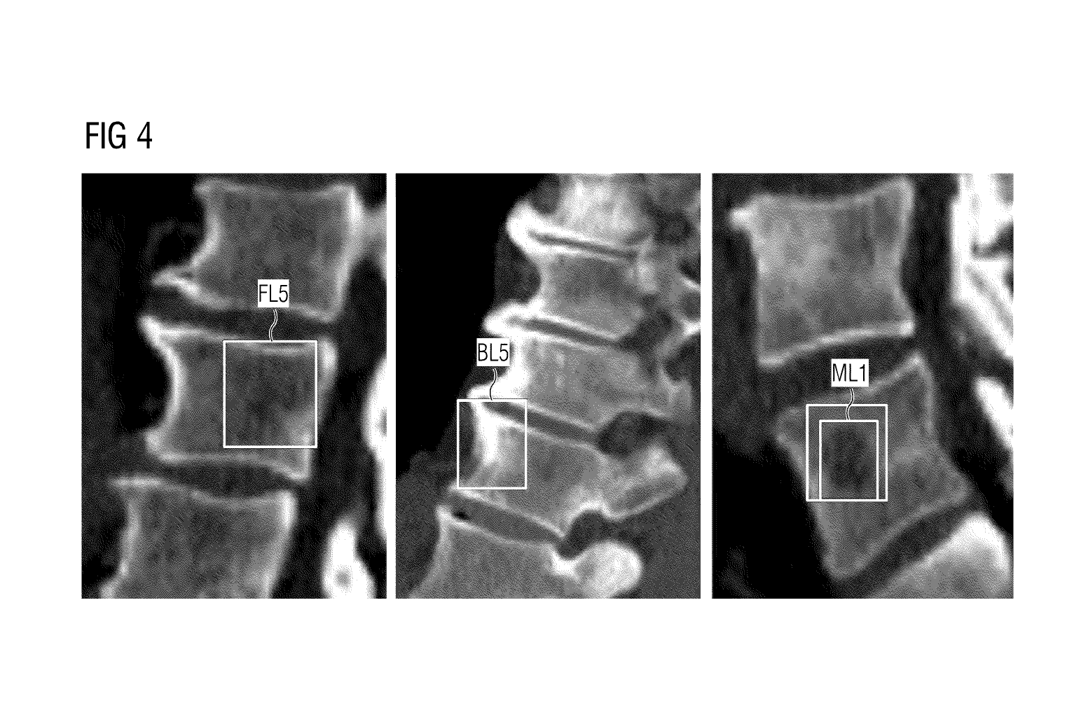

FIG. 4 shows three image data examples FL5, BL5, ML1 for rule-based classification in accordance with the method according to the invention or the image data processing workstation BS according to an embodiment of the invention. Here, the image data examples FL5, BL5, ML1 once again relate to image data BD from the region of the vertebral column. In the left-hand image data example FL5, a lesion is depicted, which was identified as a lytic-type false positive lesion FL5 by a rule-based classifier according to the invention. The lesion identification or lesion determination was in this case obtained using the following rule: “A lytic lesion is classified as a benign vertebral lesion if this is a lysis with low contrast AND if it is situated centrally in the rear plane of a vertebra”.

The central image data example BL5 in FIG. 4 depicts a lesion which was identified as a blastic-type false positive lesion BL5 by a rule-based classifier according to the invention. Here, the lesion was determined using the following rule: “A blastic lesion is classified as a benign vertebral lesion if this is a sclerotic abnormality tapering to a point AND if the lesion is situated at the edge of the vertebra”. By contrast, the right-hand image data example ML1 in FIG. 4 shows a lesion which is correctly identified as a malignant lesion ML1 by the method since none of the rules of a false positive classifier according to an embodiment of the invention can be applied thereto.

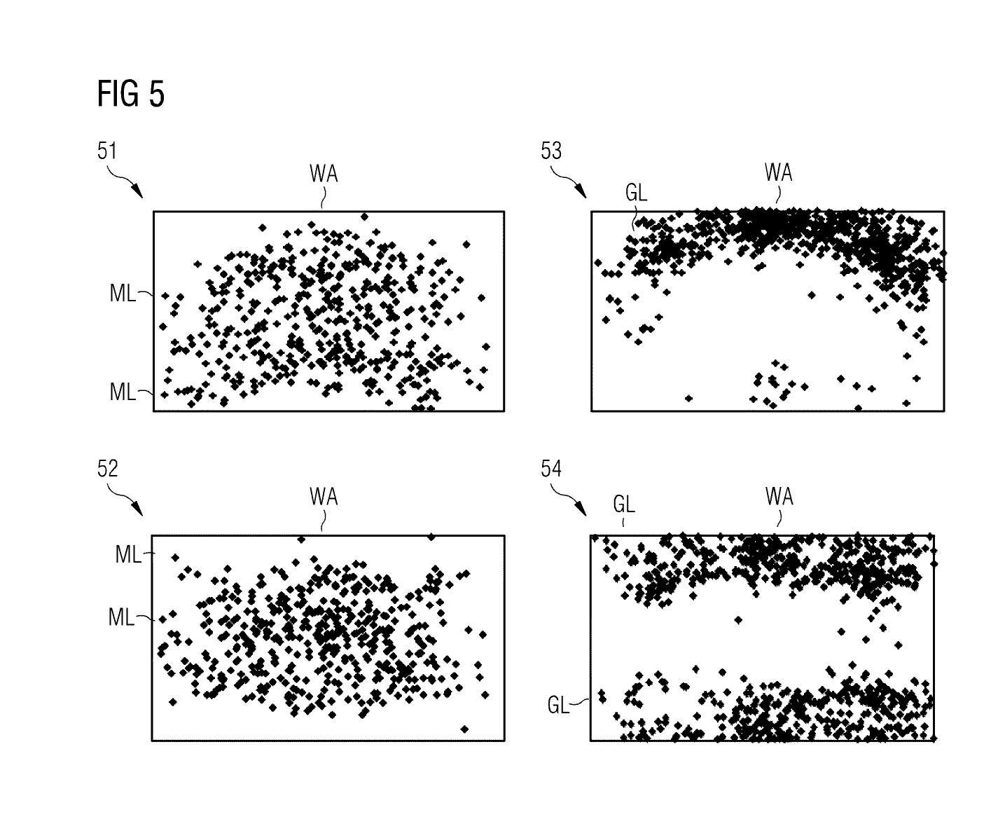

FIG. 5 depicts measurement data in respect of the spatial distribution of malignant blastic lesions ML and of benign abnormalities or lesions GL in normalized vertebrae of the vertebral column. Here, the spatial position of the lesions ML, GL is reproduced in a two-dimensional representation. Here, in all four sub-figures 51, 52, 53, 54, the rectangle WA in each case describes the contour of the normalized vertebrae, i.e. vertebrae which are scaled in terms of their dimensions to a standard rectangle or a standard edge WA. The upper two sub-figures 51, 53 show the spatial position of the lesions ML, GL in an axial view while the lower two sub-figures 52, 54 show the spatial position of the lesions ML, GL in a sagittal view. The two left-hand sub-figures 51, 52 reproduce the spatial position of malignant lesions ML, whereas the two right-hand sub-figures 53, 54 reproduce the spatial position of benign lesions GL.

It can be gathered directly from FIG. 5 that benign lesions GL such as osteophytes occur predominantly on the edge WA of a vertebra, whereas malignant lesions ML predominantly can be found in the interior of a vertebra. This clinical knowledge relating to the typical spatial distribution of lesions L advantageously can be used by the method according to the invention, in particular after suitable normalization of the anatomical structures AS, for determining the lesions L. In particular, it is possible to specify rule-based or knowledge-based classifiers, which reduce or completely avoid false positive classifications of malignant lesions ML on the edge WA of a vertebra.

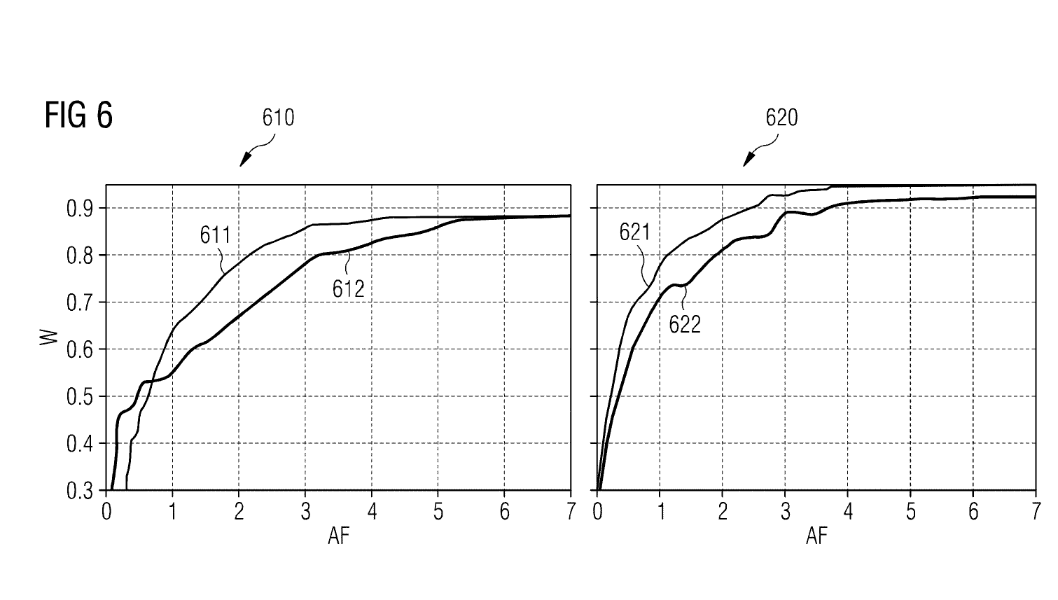

FIG. 6 shows two examples of the sensitivity W (true positive rate) as a function of the number AF of false positives per unit volume during the lesion detection. The left-hand sub-FIG. 610 here represents the profile 611, 612 of the sensitivity W for osteolytic lesions, whereas the right-hand sub-FIG. 620 reproduces the profile 621, 622 of the sensitivity W for osteoblastic lesions. Here, the curves 611 and 621 show the experimentally measured profile of the sensitivity W when using a knowledge-based classifier WB. By contrast, the curves 612 and 622 show the experimentally measured profile of the sensitivity W without the use of a knowledge-based classifier WB. Here, this is a false positive classifier which was trained by available image data from other examination objects, i.e. by training image data TB. Here, use was, in particular, also made of training image data TB of benign abnormalities as representatives for false positive lesions.

It can be gathered directly from FIG. 6 that the probability of incorrect and undesired false positive classifications of malignant lesions can be substantially reduced by the use according to an embodiment of the invention of the knowledge-based classifier WB. In addition to the rule-based classifiers described above, which take into account the spatial position of the lesions L and, in particular, are suitable for determining benign osteophytes, knowledge-based classifiers WB are found to be suitable for separating malignant lytic lesions from benign basivertebral veins or benign osteoporosis. Moreover, such knowledge-based classifiers WB are suitable for separating between malignant blastic lesions and benign degenerative abnormalities.

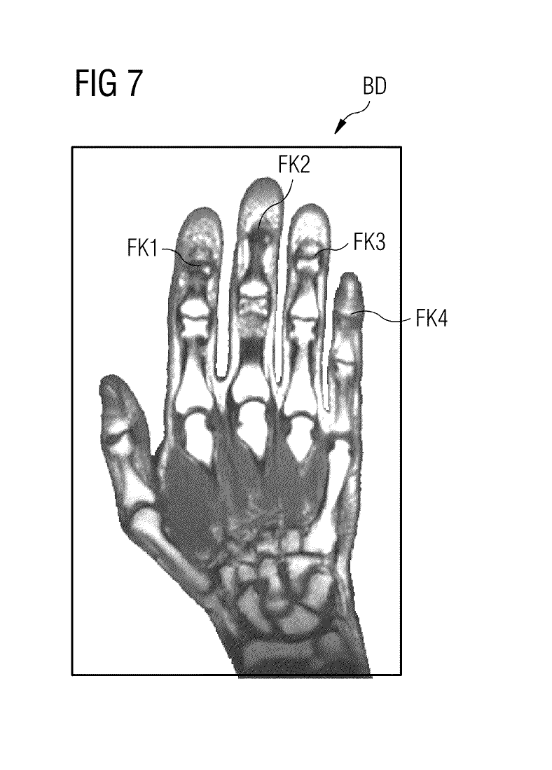

In addition to the vertebrae of the vertebral column of a patient, numerous other anatomical structures AS of the human are also suitable for an advantageous normalization within the method according to an embodiment of the invention. In an exemplary manner, FIG. 7 shows a group of four phalanges FK1, FK2, FK3, FK4 in the image data BD of a hand, which are suitable for such a normalization, for example by scaling, rotation, grayscale value adaptation and bone density standardization. Here, such a normalization can in each case also include the phalanges FK1, FK2, FK3, FK4 of the corresponding phalanges group of the second hand of the patient. Optionally, the normalization can also be extended to further groups of phalanges in the hand or extended by a common normalization with the phalanges in the foot.

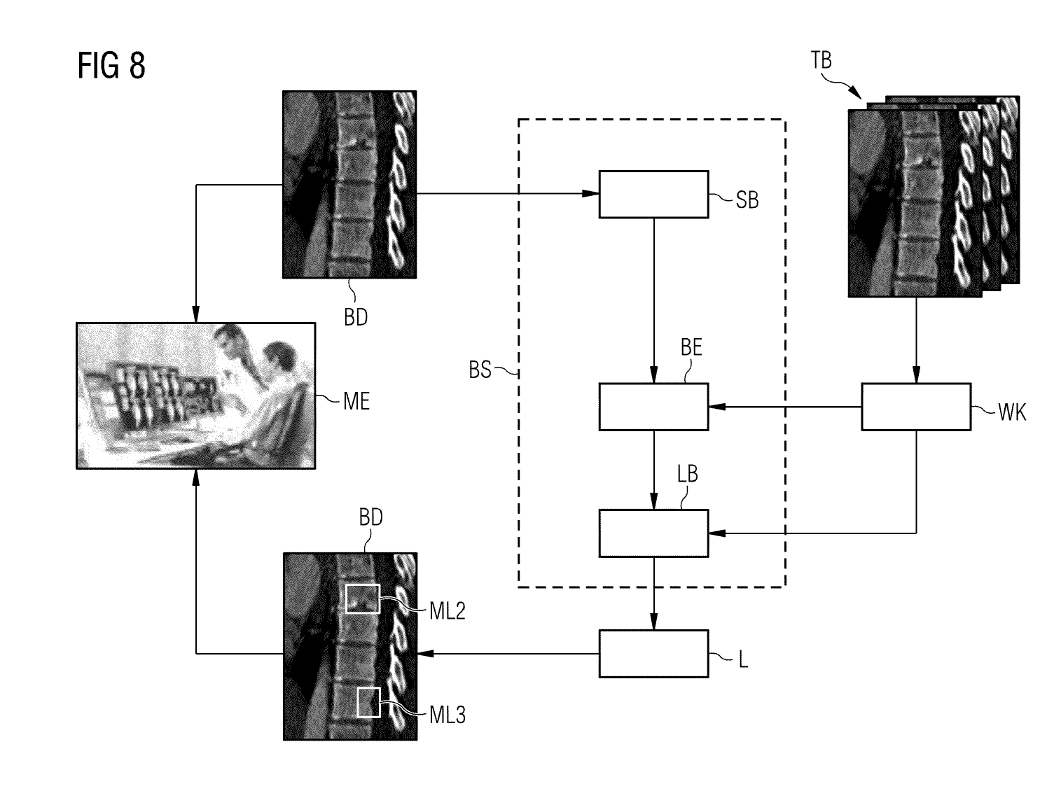

FIG. 8 shows an embodiment of the image-processing workstation BS according to the invention, which determines lesions L in the image data BD of the examination object. The image-processing workstation BS has a structure determination apparatus SB for determining anatomical structures AS by hierarchical breakdown HZ of the image data BD of the examination object. Furthermore, the image-processing workstation BS comprises an image data analysis apparatus BE for localizing lesion candidates LK in the anatomical structures AS, and a lesion determination apparatus LB for determining the lesions L by evaluating and filtering BF the lesion candidates LK. Here, both the image data analysis apparatus BE and the lesion determination apparatus LB can be controlled by knowledge-based classifiers WB, which were trained by other image data TB. The malignant lesions ML1, ML2 determined by the image data processing workstation BS can subsequently be marked in the image data BD, for example by a rectangle, such that they are available for further analysis and evaluation by a medical expert ME.

To conclude, reference is once again made to the fact that the methods and image-processing workstations described in detail above are merely exemplary embodiments, which can be modified by a person skilled in the art in very different ways, without departing from the scope of the invention. In particular, the embodiments of the method according to the invention can advantageously be employed e.g. not only for the vertebral column body region but also in the radiological imaging of other body regions.

For the sake of completeness, reference is also made to the fact that the use of the indefinite article “a” or “an” does not preclude the possibility of the relevant features also being present a number of times.

1. A method in radiological imaging for determining lesions in image data of an examination object, comprising:
determining anatomical structures by hierarchical breakdown of the image data of the examination object;
analyzing image data for localizing lesion candidates in the anatomical structures; and
determining the lesions by evaluating and filtering the lesion candidates.

2. The method of claim 1, wherein at least one of the localizing of the lesion candidates and the determining of the lesions is controlled by specific features of the anatomical structures.

3. The method of claim 1, wherein a number of rule-based classifiers are used for at least one of localizing the lesion candidates and determining the lesions.

4. The method of claim 3, wherein the number of rule-based classifiers comprises a number of false positive classifiers.

5. The method of claim 1, wherein, for determining the anatomical structures, at least one of a number of anatomical atlases are used and a number of anatomical landmarks are determined in the image data.

6. The method of claim 1, wherein analyzing image data for localizing lesion candidates is predominantly carried out on the basis of at least one of pixels and voxels.

7. The method of claim 1, further comprising normalizing the anatomical structures.

8. The method of claim 1, wherein the method is controlled by specific features of the examination object.

9. The method of claim 1, wherein the determining of the lesions comprises a separating classification of the lesions into benign lesions and malignant lesions.

10. The method of claim 1, wherein the image data of the examination object comprise a whole-body image data record.

11. The method of claim 1, wherein the method determines lesions in the skeleton of the examination object.

12. The method of claim 1, wherein the determining of the lesions comprises a separating classification of the lesions into blastic and lytic lesions.

13. An image-processing workstation in radiological imaging for determining lesions in image data of an examination object, comprising:
a structure determination apparatus, configured to determine anatomical structures by hierarchical breakdown of the image data of the examination object;
an image data analysis apparatus, configured to localize lesion candidates in the anatomical structures; and
a lesion determination apparatus, configured to determine the lesions by evaluating and filtering the lesion candidates.

14. An imaging apparatus, comprising the image-processing workstation of claim 13.

15. A computer program product, loadable directly into a memory of a programmable imaging apparatus, comprising program code segments for executing the method of claim 1, when the program is executed in the imaging apparatus.

16. The method of claim 3, wherein the number of rule-based classifiers are a number of knowledge-based classifiers trained using image data of other examination objects.

17. The method of claim 2, wherein a number of rule-based classifiers are used for at least one of localizing the lesion candidates and determining the lesions.

18. The method of claim 4, wherein the number of false positive classifiers are knowledge-based false positive classifiers trained by false positive image data from other examination objects.

19. The method of claim 11, wherein the method determines lesions in the vertebral column of the examination object.

20. A computer program product, loadable directly into a memory of a programmable imaging apparatus, comprising program code segments for executing the method of claim 2, when the program is executed in the imaging apparatus.
